(19) United States
(12) Patent Application Publication
(10) Pub. No.: US 2007/0016089 A1
(43) Pub. Date: Jan. 18, 2007

(54) Implantable device for vital signs monitoring

(76) Inventors: David R. Fischell, Fair Haven, NJ (US); Tim A. Fischell, Kalamazoo, MI (US); Jonathan Harwood, Rumson, NJ (US); Steven R. Johnson, Fair Haven, NJ (US); Gregg Turi, Mount Olive, NJ (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/181,969

(22) Filed: Jul. 15, 2005

(51) Int. Cl. A61B 5/04; A61B 5/00; A61B 5/02

(52) U.S. Cl. 600/509; 128/903; 600/323; 600/549; 600/486; 600/521

(57) ABSTRACT

An implantable medical device is provided for subcutaneous implantation within a human being. The implantable medical device includes a pair of electrodes for sensing electrical signals from the human being's heart. Electronic circuitry having digital memory is provided with the electronic circuitry designed to record the electrical signals from the heart. The electronics of the electronic circuitry are housed in a case having a tapered shape to facilitate implantation and removal of the implantable medical device.

### FIELD OF USE

This invention is in the field of devices implanted within a human patient having the ability to measure vital signs of a human patient.

### BACKGROUND OF THE INVENTION

In U.S. Pat. No. 6,609,023 which is incorporated herein by reference, Fischell et al describe a system implanted like a pacemaker for the detection of cardiac events with patient alerting. Although the Fischell system describes the measurement of heart signal parameters from a subcutaneously implanted device, the Fischell system is not designed to measure other vital signs including temperature, blood oxygen, blood pressure, patient activity and autonomic nervous system balance. In U.S. Pat. No. 5,987,352, Klein et al describe a subcutaneous implantable loop recorder having fixed electrodes on the outer surface of the housing. Such fixed electrodes limit the quality of received signals because the spacing is limited by the size of the housing. Also the shape of the Klein device is not tapered and has substantially uniform width and thickness. The Reveal Plus™ by Medtronic, which relates to the Klein patent, is a subcutaneously implantable loop recorder which has limited ability to detect syncope and bradycardia but has no measurement capability beyond simple recording of electrocardiogram data. It is also limited to 42 minutes of data recording and cannot function as a 24 hour Holter monitor. In U.S. Pat. No. 5,313,953, Yomtov et al describe an implantable cardiac monitor with a subcutaneous lead which is similar in shape to a pacemaker and is only designed to process electrocardiogram signals. External Holter monitoring devices which can record electrocardiogram data for 24 or more hours require external attachment of electrodes to the patient's skin and are not useful for patient monitoring for extended periods of time. The Chronicle device from Medtronic collects electrogram and blood pressure information but requires a ventricular lead that extends into the heart. The Rheos™ Baroreflex Hypertension Therapy System™ by CVRx (U.S. Pat. Nos. 6,522,926, 6,616,624 and 6,850,801) utilizes an electrical signal from the carotid sinus to measure blood pressure but does not include electrocardiogram signal analysis or other vital signs.

### SUMMARY OF THE INVENTION

The present invention system is a vital signs monitoring system including a subcutaneously implanted Vital Signs Monitor (VSM) an EXternal Device (EXD) and a physician's programmer.

The implantable vital signs monitor includes leads of different lengths that are attachable to a shell housing having an exterior and an interior, the interior of the shell housing including a battery and electronic circuitry. The vital signs monitor shell housing including a single sensing electrode on the outer surface of the exterior of the shell housing. The vital signs monitor lead may be extremely short (lead less than 1 inch long) that includes a single electrode that works with the single electrode on the exterior of the shell housing to sense electrocardiogram signals from the heart. This short lead may be substantially more rigid than a longer lead.

For better signal quality including the detection of P-waves, an intermediate length lead between 1 and 4 inches long may be desirable. The intermediate length lead may be comparable to a pacing lead physical characteristic or may be substantially more rigid (yet remaining soft/flexible enough to prevent extrusion in the body). The soft/flexible lead would maintain electrode spacing better with fewer suture requirements. The intermediate length lead can have one or more electrodes spaced along its length and preferably a shape or suture means that will prevent the electrode on the intermediate lead from moving with respect to the electrode on the outer surface of the control module.

For even better signal quality including the measurement of ST segment levels as described by Fischell et al in U.S. Pat. No. 6,609,023 a long subcutaneous lead greater than four inches long is desirable. If the vital signs monitor includes a standard lead interface such as an IS-1 interface, then for the best signal quality it is envisioned that the vital signs monitor can be attached to a standard intra-cardiac lead such as the 1488T steroid eluting right ventricular lead from St. Jude Medical. In this way depending on the need of the patient, the physician can implant the vital signs monitor with the appropriate lead and even change the lead if enhanced signal quality is needed.

The VSM electronics could have the ability to use different band pass filters for different applications and lead configurations. The band pass response could be programmable or could be automatically selected based on coding information within a specific lead configuration when attached to the device. For example with the shortest lead where R-wave timing is of primary importance a 1 Hz or 2 Hz low pass filter cut-off would be fine. For the longer lead configuration, where ST segment levels are to be measured, a lower cut-off of less than 1 Hz (ideally 0.25 Hz to 0.5 Hz) is required. Appropriate filtering for ST segment level measurement in implantable devices is described by Fischell et al in U.S. patent application Ser. No. 10/741,141 which is incorporated herein by reference.

It is also envisioned that the vital signs monitor would include at least one temperature sensor and software to measure and track patient temperature over an extended period of time. An accelerometer would also be included to monitor patient activity for correlation with other vital signs including heart rate, temperature and other heart signal parameters.

An important sensor for the vital signs monitor is a Photo-PlesmoGraphy (PPG) sensor, (an optical sensor) which measures the oxygen content in the vascular bed under the implanted vital signs monitor. PPG sensors are described in U.S. Pat. No. 6,491,639 by Turcott which is incorporated herein by reference. The PPG signal can be recorded continuously or periodically to conserve power. Analysis of the PPG signal can take place within the VSM, EXD or physician's programmer and can be used to identify changes in the patient's cardiovascular condition or respiratory function. Such analysis by the VSM or EXD may include the use of histograms to provide significant data compression. It is also envisioned that the effect of heart rate on the PPG signal may be part of the analysis by the VSM or EXD. Specifically effects on the systolic/diastolic differences in the PPG signal as heart rate increases may indicate progression of cardiovascular disease or congestive heart failure long before other symptoms appear. One embodiment of the PPG signal analysis will track the median systolic/diastolic difference in signal over an extended period of time. The histogram techniques of U.S. patent application Ser. No. 10/950,401 by Fischell et al are applicable to the PPG signal as well the electrocardiogram signal. PPG signals can also be used by the vital signs monitor to diagnose episodes of sleep apnea, pulmonary edema, heart failure decompensatoin and asthma. PPG can also be used to monitor pulmonary function, tidal volume, vascular tone, arterial and venous oxygen saturation, heart failure, respiration rate and heart rate. The present invention VSM expands upon Turcott's PPG system through use of low power drain half wave analysis to track changes in heart and lung function.

The electrocardiogram recording and analysis provided by the VSM includes loop recording and/or electrocardiogram segment recording capabilities either patient initiated using the EXD or initiated by the detection by the VSM of one or more health related events including but not limited to:
1. Syncope
2. Bradycardia
3. Tachycardia
4. Atrial Fibrillation
5. Atrial Flutter
6. Premature Ventricular Contractions (PVCs)
7. Premature Atrial Contractions (PACs)
8. ST Elevation
9. ST Depression
10. QRS width changes
11. T wave Alternans
12. Changes in RR Interval Variability
13. Bigeminal and Trigeminal Rhythms
14. AV node dysfunctions
15. Winkybach arrhythmias
16. Wandering P waves
17. Wolff-Parkinson-White syndrome
18. High or low blood pressure
19. Change in R-R interval variability
20. Fever
21. Hypothermia
22. Low blood oxygen levels

Loop recording would be done in a way similar to the Medtronic Reveal and strip recording would be done in a way similar to the cardiosaver and cardiotracker devices of the Fischell patents and patent applications referenced above. The VSM could also provide periodic strip (electrocardiogram segment) recording. For example a 10 second electrocardiogram segment or strip could be recorded and saved once per hour and offloaded to the EXD memory periodically, for example, once per day.

The VSM is also designed to have a unique tapered shape that facilitates implantation through a small slit in the patient's skin of approximately ⅝′. The tapered shape includes tapering in both thickness and housing width with a curved shape somewhat like a banana or boomerang.

There are a number of different ways in which the VSM can include blood pressure measurements as part of its vital signs monitoring. If a lead is implanted into the heart, a sensor on the lead may be used. Another technique is to process the PPG signal to provide short term changes in blood pressure. The RheosTm Baroreflex Hypertension Therapy System™ by CVRx utilizes an electrical signal from carotid sinus leads to measure blood pressure. Such leads could be connected to the VSM for monitoring blood pressure. Finally, there are numerous automated blood pressure cuffs for arms, wrists or fingers that could be worn by the patients and enhanced to allow wireless communication of blood pressure information to the VSM or EXD for storage and analysis.

The present invention VSM and EXD would utilize the state of the art in long range (up to 10 feet) telemetry using chipsets such as the Ash hybrids of RF microdevices or the ChipCon CC1000/CC1020 chipset. The antenna for the long range telemetry can be in the lead, a patch antenna on the surface of the housing, in the header where the lead connects or a separate antenna extending outside of the housing. The preferred embodiment of the EXD would have a rechargeable battery, have one or more buttons and be insertable into a recharging cradle with connection to a phone line, Ethernet, wireless (802.11 or Bluetooth) for data downloading to the patient's doctor or a data collection service. Enhancements on the EXD would add cellular voice/data connectivity, a beeper, voice messages, LEDs and/or a text/graphic display for patient communication, GPS and alerting. The EXD could also have means to insert a standard USB thumbdrive and/or flash memory card. Examples of standard flash memory cards are compact flash cards, secure digital cards and memory sticks.

It is also envisioned that the VSM and/or lead (except for the electrode) may be coated with a biodegradable or non-biodegradable material to provide an antibacterial or drug eluting layer to prevent infection related to the implant.

The VSM may also have a built-in patient alerting capability using sound, vibration or an electrical tickle.

Thus it is an object of this invention to have an implantable vital signs monitor having a shape that is tapered in either or both width and thickness for improved insertion.

Another object of the present invention is to have a vital signs monitor that includes temperature, blood pressure, electrocardiogram and Photo-PlesmoGraphy signal storage.

Still another object of this invention is to have a vital signs monitor with a single electrode on the outer surface of the electronics shell housing and a second electrode on an attachable lead.

Yet another object of the present invention is have a vital signs monitor including attachable leads of two or more lengths.

Yet another object of the present invention is to utilize the PPG signal as a function of heart rate to track cardiovascular condition of a human patient.

Yet another object of the present invention is to have a vital signs monitor utilize a programmable mode to allow information transfer of data and parametric measurements to facilitate a Holter monitoring (with data being accumulated in the EXD).

Yet another object of the present invention is to have a vital signs monitor provide an alerting function (either or both internally with the VSM and externally by the EXD) in the form of either sound, vibration, or subcutaneous electrical stimulation (tickle).

Yet another object of the present invention is to have a vital signs monitor which includes programmable data analysis of monitored parameters, programmable trending/histogram information of monitored parameters, programmable automatic triggering of monitored parameters, and programmable alerting of monitored parameters.

Yet another object of the present invention is to have a vital signs monitor with extensive programmability with regard to automatic triggering capability based on absolute values, relative shifts, trends, etc., including multiple scenario trigger conditions (i.e. perform autotrigger action X if heart rate determined to be Bradycardia, and perform autotrigger action Y if heart rate is tachycardia, etc.).

Yet another object of the present invention is to have a vital signs monitor that includes blood pressure monitoring.

These and other objects and advantages of this invention will become obvious to a person of ordinary skill in this art upon reading of the detailed description of this invention including the associated drawings as presented herein.

### BRIEF DESCRIPTION OF THE DRAWINGS

FIG. 1 illustrates the prior art Medtronic Reveal™ implantable loop recorder.

FIG. 2 illustrates a side view of the present invention vital signs monitor including a short lead.

FIG. 3 illustrates a top view of the vital signs monitor of FIG. 2.

FIG. 4A illustrates a side view of an alternate embodiment of the present invention vital signs monitor.

FIG. 4B illustrates the other side view of the vital signs monitor of FIG. 4A.

FIG. 5 illustrates a top view of the vital signs monitor of FIGS. 4A and 4B.

FIG. 6 is a block diagram of the vital signs monitor system detailing the components of the implantable vital signs monitor.

FIG. 7 is a block diagram of the vital signs monitor system detailing the internal components of the external device (EXD).

FIG. 8 shows a 20 second trace of the PPG signal which is the output of the PhotoPlesmoGraphy (PPG) sensor of FIG. 6.

FIG. 9A illustrates the vital signs monitor of FIGS. 4A, 4B and 5 with a very short lead.

FIG. 9B illustrates the vital signs monitor of FIGS. 4A, 4B and 5 ith a mid length lead suitable for P wave detection.

FIG. 9C illustrates the vital signs monitor of FIGS. 4A, 4B and 5 with a long length lead suitable for ST segment analysis and ischemia detection.

FIG. 10 is an example of an R-R interval variability spectrum showing low-frequency, mid-frequency and high-frequency peaks.

### DETAILED DESCRIPTION OF THE INVENTION

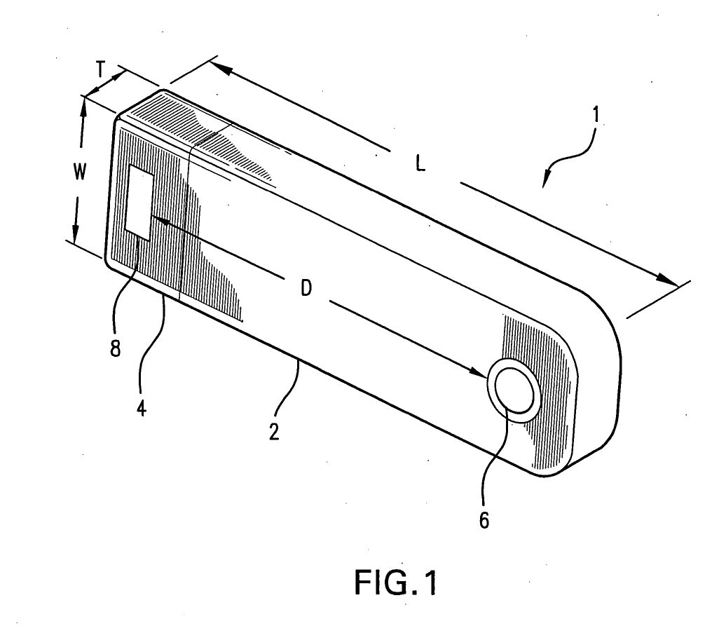

FIG. 1 illustrates the prior art implantable loop recorder 1 having a shell housing 2 with electrode 6 on the outside of the shell housing. The implantable loop recorder 1 also has a plastic header 4 with electrode 8 on its outer surface. The electrodes 6 and 8 are separated by a fixed distance “D”. The shape of the implantable loop recorder 1 is designed to be placed subcutaneously through a small slit in the skin. In spite of this, the shape of the implantable loop recorder 1 is of substantially uniform width “W” and thickness “T” although the width “W” is less than the length “L”. The prior art implantable loop recorder 1 which is sold as the Medtronic Reveal™ implantable loop recorder is designed to record electrical signals from the heart with a total recording time of up to 42 minutes. Recording is continuous with the data captured for later physician review by patient initiation using an external device or an event detected by the implantable loop recorder. The implantable loop recorder 1 is designed to detect Syncope, Tachycardia and Bradycardia so as to help diagnose rarely occurring abnormal heart activity.

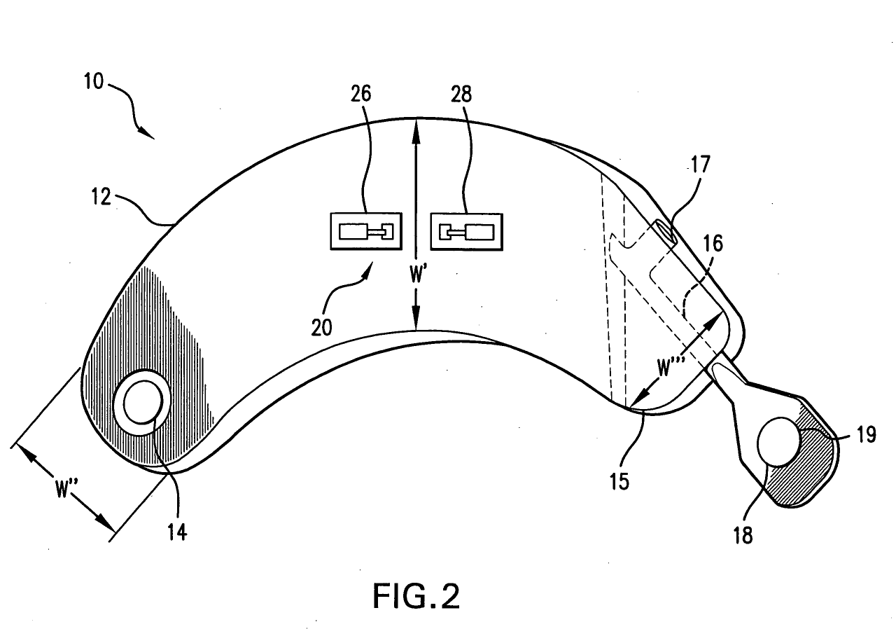

FIG. 2 illustrates a view of the back side of a first embodiment of the present invention implantable vital signs monitor (VSM) 10. The VSM 10 has a shell housing 12 with electrode 14 on the surface of the shell housing 12. A header 15 with a lead interface 16 with securing set screw 17 is designed to allow insertion of the lead 18 with electrode 19. Unlike the prior art implantable loop recorder 1 of FIG. 1, the electrode spacing can be changed by use of leads of different lengths. Specifically, the short lead 18 typically less than 1″ long is well suited to detecting arrhythmias related to measuring the R waves in the electrocardiogram. Longer leads as described in the discussion which follows can be attached to the VSM to enable the detection of P wave and ST segment related abnormalities.

The shape of the VSM 10 is designed to facilitate subcutaneous insertion in the pectoral region where pacemakers and the implantable loop recorder 1 of FIG. 1 are typically implanted. Specifically, the VSM 10 is tapered in both width and thickness and has a curved shape. The curved shape may fit better around the breast area of either a man or woman than the straight design of the prior art VSM 1 of FIG. 1. The tapered width of the VSM 10 is seen by the variation in the width from the width W′ in the center of the shell housing 12 tapering to the width W″ at the end of the shell housing with the electrode 14 and a width W′″ at the end of the header 15 where the lead is inserted into the lead interface 16. Thus W′ is larger than the widths W″ and W′″ allowing improved insertion through a slit in the skin as well as device removal following diagnosis of the patient's condition. The tapered width is an advantage of the present invention of the VSM 10 over the prior art implantable loop recorder 1 of FIG. 1.

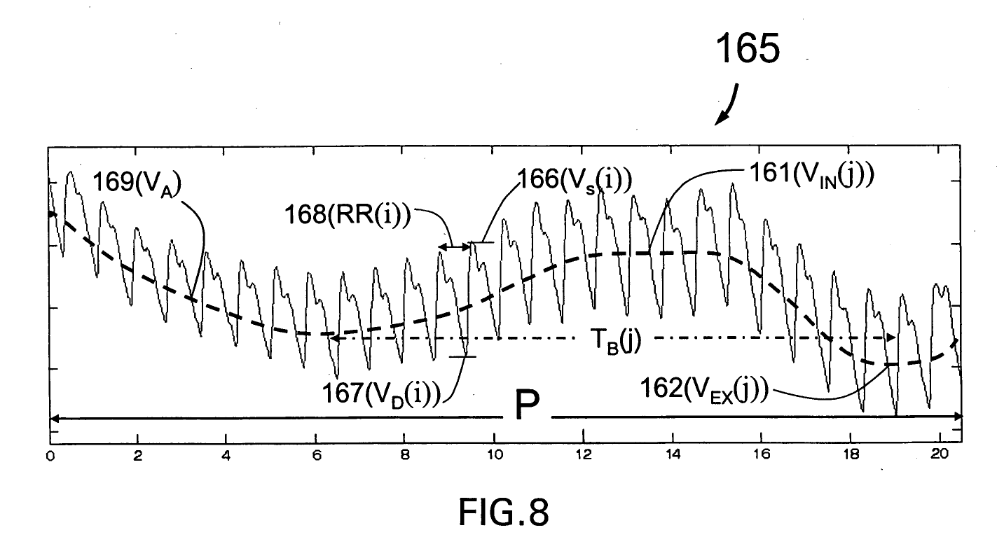

Mounted in the side of the VSM 10 is the PhotoPlesmoGraphy (PPG) sensor 20 having light source 26 and detector 28. The PPG sensor 20 is well described as an extrovascular hemodynamic sensor by Turcott in U.S. Pat. No. 6,491,639 which is incorporated herein by reference. Turcott envisions the PPG sensor as part of a pacemaker or implantable cardioverter defibrillator (ICD) having one or more intracardiac leads. The PPG sensor 20 greatly enhances the capabilities of the VSM 10 by providing blood oxygen data that can track both heart and lung function. Application of the PPG sensor 20 to vital signs monitoring will be described in greater detail in the description of FIG. 8 which follows. While the VSM 10 is typically implanted with the electrodes 14 and 19 as well as the PPG sensor 20 facing down toward the patient's heart the VSM would also function, although not as well, with either or both the electrodes 14 and 19, and PPG sensor 20 facing outward toward the patient's skin.

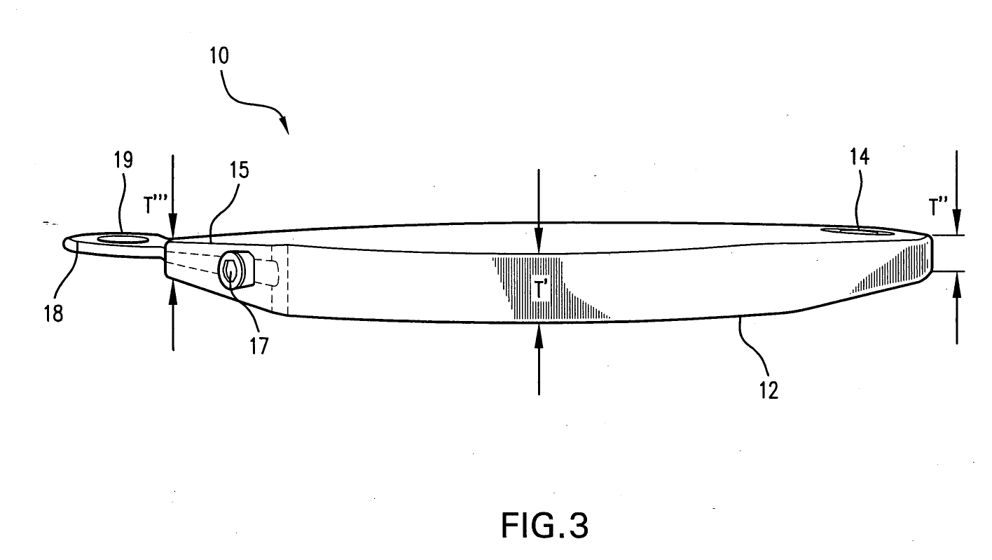

FIG. 3 illustrates a top view of the VSM 10 of FIG. 2 showing the shell housing 12 with electrode 14, header 15 with set screw 17 and lead 18 with electrode 19. The VSM 10 is tapered in thickness to improve subcutaneous insertion. Specifically the thickness of the VSM 10 in the center of the shell housing 12 T′ is greater than the thickness T″ at the end of the shell housing 12 having the electrode 14. The thickness T′ is also greater than the thickness T′″ of the header 15 at the end where the lead 18 is inserted.

While the VSM 10 shown in FIGS. 2 and 3 is tapered in both width and thickness it is envisioned that the present invention VSM may be of substantially uniform thickness but tapered in width or of substantially uniform width but tapered in thickness. The VSM 10 header 15 has one lead attachment screw 17, which would typically indicate the VSM is designed to interface to a monopolar lead. It is also envisioned that the lead interface 16 shown in FIG. 2, could be designed to interface to bipolar or multipolar leads. The lead interface 16 may be unique to the VSM 10 and associated leads or it could be a standardized lead interface such as an IS-1 or IS-4 interface. It is also envisioned that the VSM 10 header 15 could include multiple lead interfaces to facilitate connection of multiple leads.

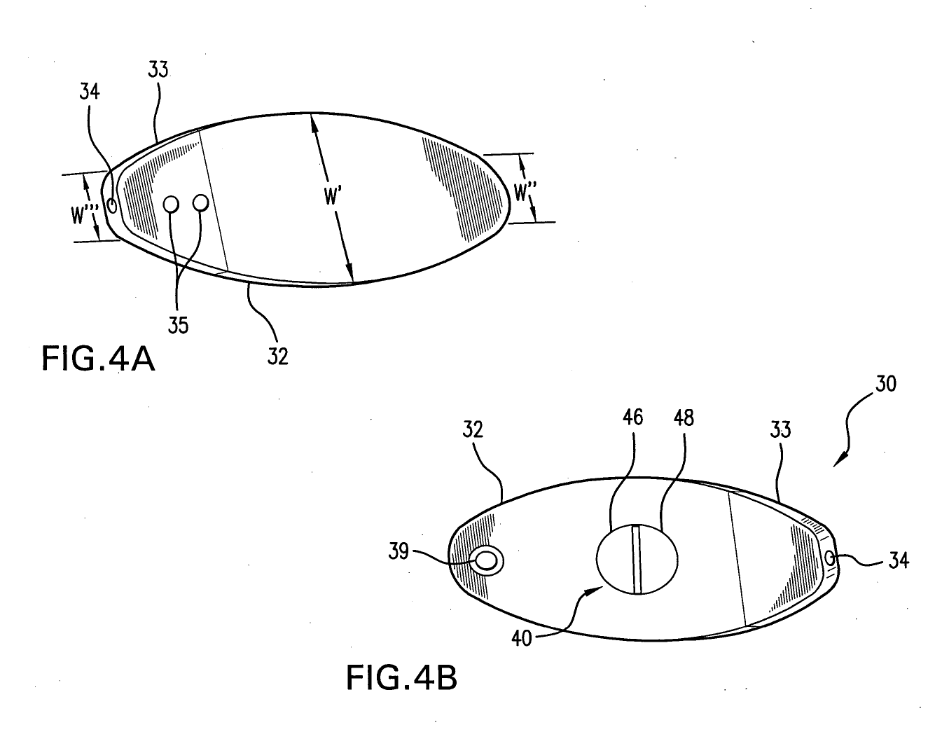

FIG. 4A and 4B illustrate front (4A) and back (4B) sides of an alternate embodiment of the present invention vital signs monitor (VSM) 30. The VSM 30 has straight tapered shape different from the curved tapered shape of the VSM 10 of FIG. 2. The VSM 30 includes a shell housing 32 with electrode 39, header 33 with lead interface 34 and set screws 35 for securing the lead (not shown) into the lead interface 34. Two set screws 35 as shown in FIG. 4A indicate that the VSM 30 can interface to a lead with two electrodes (e.g. a bipolar lead). The tapered width of the VSM 30 is seen by the variation in the width from the width w′ in the center of the shell housing 32 tapering to the width w″ at the end of the shell housing with the electrode 39 and a width w′″ at the end of the header 15 where the lead is inserted into the lead interface 34. Thus w′ is larger than the widths w″ and w′″ allowing improved insertion through a slit in the skin as well as device removal following diagnosis of the patient's condition. The tapered width is an advantage of the present invention of the VSM 30 over the prior art implantable loop recorder 1 of FIG. 1.

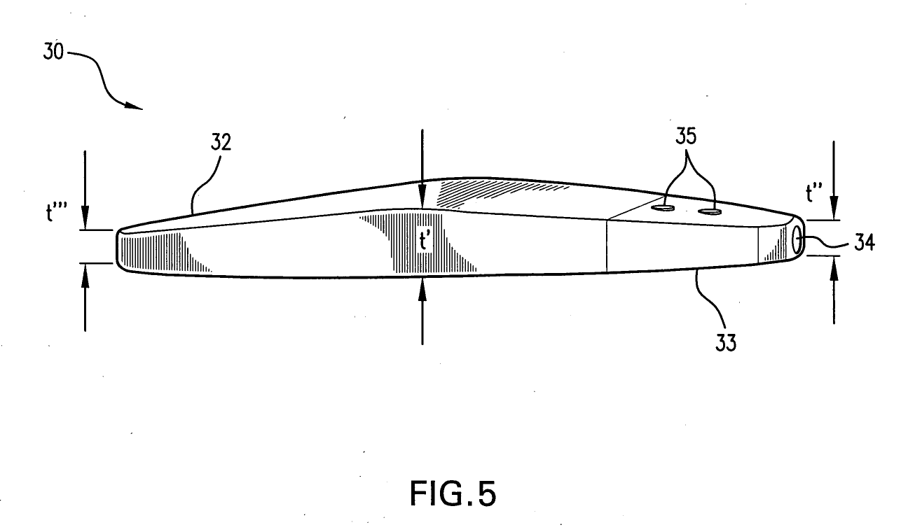

While the VSM 30 shown in FIGS. 4A, 4B and 5 is tapered in both width and thickness it is envisioned that the present invention VSM may be of substantially uniform thickness but tapered in width or of substantially uniform width but tapered in thickness. The VSM 30 header 33 has two lead attachment screws 35, which would typically indicate the VSM is designed to interface to a bipolar or monopolar lead. It is also envisioned that the lead interface 34 could be designed to interface to a multipolar leads (i.e. leads with more than 2 electrodes). The lead interface 34 may be unique to the VSM 30 and associated leads or it could be a standardized lead interface such as an IS-1 or IS-4 interface. It is also envisioned that the VSM 30 header 33 could include multiple lead interfaces to facilitate connection of multiple leads.

Mounted in the side of the VSM 30 is the PhotoPlesmoGraphy (PPG) sensor 40 having light source 46 and detector 48. While the VSM 30 is typically implanted with the electrodes 39 as well as the PPG sensor 40 facing down toward the patient's heart the VSM would also function, although not as well, with the electrode 39, and PPG sensor 40 facing outward toward the patient's skin.

FIG. 5 illustrates a top view of the VSM 30 of FIG. 2 showing the shell housing 32, header 33 with set screws 35 and lead interface 34. The VSM 30 is tapered in thickness to improve subcutaneous insertion. Specifically the thickness t′ of the VSM 30 in the center of the shell housing 32 is greater than the thickness t″ at the end of the shell housing 32 having the electrode 39. The thickness t′ is also greater than the thickness t′″ of the header 33 at the end where the lead is inserted.

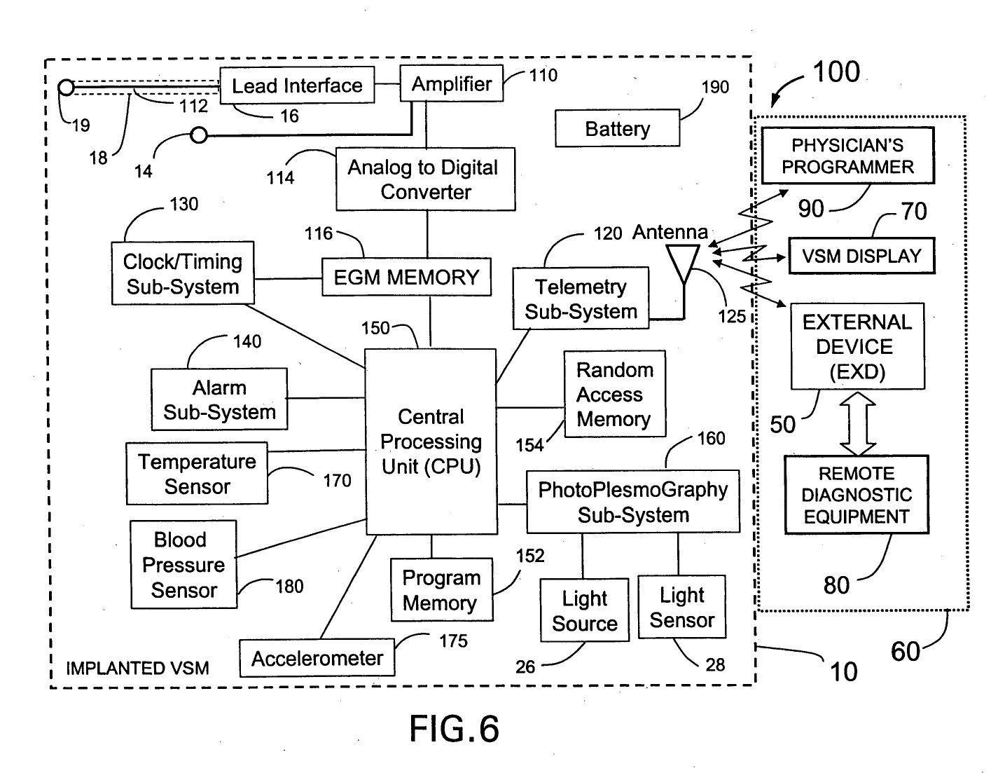

FIG. 6 is a block diagram of a vital signs monitoring system 100 including an implanted vital signs monitor (VSM) 10, an external device (EXD) 50, a vital signs monitor display (VSM Display) 70 and remote diagnostic equipment 80. FIG. 6 shows the components of the VSM 10 of FIGS. 2 and 3 and also applies to alternate embodiments such as the VSM 30 of FIGS. 4A 4B and 5.

The VSM 10 is powered by the battery 190 and is controlled by the CPU 150 having program memory 152 and random access memory 154. Electrical signals from the heart (electrocardiogram signals) are sensed with the electrodes 14 and 19. The electrode 19 being part of the lead 18 having conductor 112 that connects the lead 19 to the lead interface 16 which in turn is connected to the amplifier 110. The amplified signals from the amplifier 110 are then digitized by the analog to digital converter 114. The now digitized signals are then written to the electrogram storage memory (EGM Memory) 116. The CPU 150 can transfer all or part of the electrogram storage memory to the random access memory space 154 to be saved for later review. The CPU 150 can also freeze the electrogram storage memory 116 so that all or part of the electrogram storage memory 116 is no longer overwritten by new digitized signals from the analog-to-digital converter 114. The CPU 150 can also transfer the signals stored in the electrogram storage memory through the telemetry sub-system 120 with antenna 125 to external equipment 60 including the external device (EXD) 50, the physician's programmer 90 and/or the vital signs monitor display 70. The CPU 50 may also process the electrical signals from the heart stored in the electrogram storage memory 116. This processing includes the detection of cardiac events and the extraction of various parameters from the electrocardiogram signal.

It is also envisioned that the program memory 152, electrogram storage memory 116 and/or random access memory 154 can be physically separate or reside within the same memory circuits or chips.

Health related events (including cardiac events) that may be detected by the VSM 10 include but are not limited to:
1. Syncope
2. Bradycardia
3. Tachycardia
4. Atrial Fibrillation
5. Atrial Flutter
6. Premature Ventricular Contractions (PVCs)
7. Premature Atrial Contractions (PACs)
8. ST Elevation
9. ST Depression
10. QRS width changes
11. T wave Altemans
12. Changes in RR Interval Variability
13. Bigeminal and Trigeminal Rhythms
14. AV node dysfunctions
15. Winkybach arrhythmias
16. Wandering P waves
17. Wolff-Parkinson-White syndrome
18. High or low blood pressure
19. Change in R-R interval variability
20. Fever
21. Hypothermia
22. Low blood oxygen levels

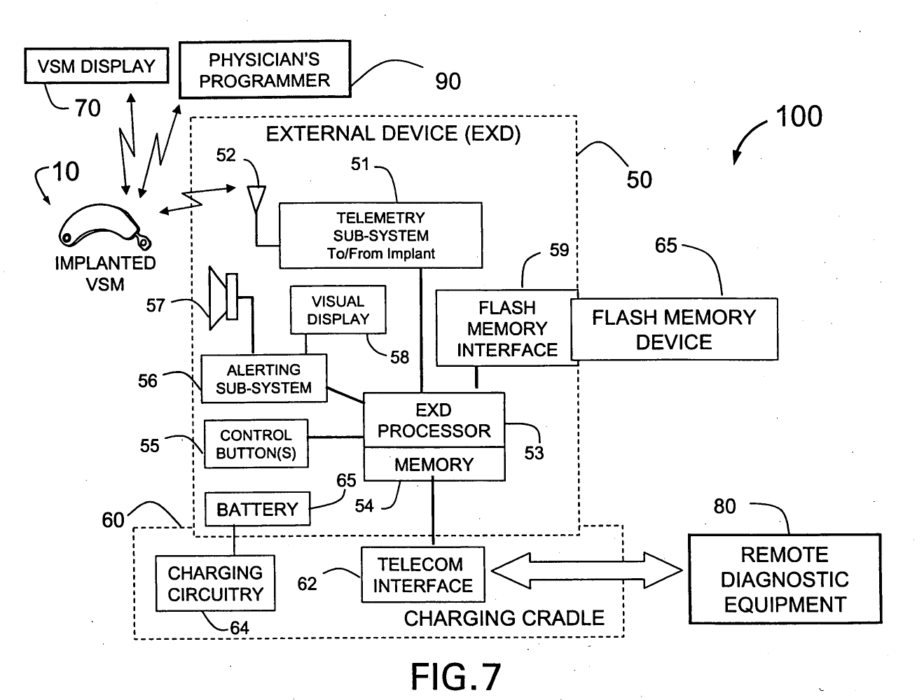

The VSM 10 is designed to retain in memory for physician review, the specific data leading up to and following the detection of a cardiac event. The ability to transfer data through the telemetry sub-system 120 greatly increases the amount of such data retained by the VSM system 100. Like the prior art loop recorder of FIG. 1, the external device (EXD) 50 allows the patient to initiate the capture and retention of electrocardiogram signals which may be stored for later physician review in the electrogram storage memory 116, the random access memory 154 or transferred to the EXD 50 whose structure is shown in FIG. 7.

Although the program memory 152, electrogram storage memory 116 and random access memory 154 are shown as separate in FIG. 6 they may in fact be located within the same memory component of the VSM 10.

The VSM 10 also includes several other vital signs sensors. These include a temperature sensor 170, a blood pressure sensor 180 and a PhotoPlesmoGraphy (PPG) sub-system 160 with light source 26 and sensor 28. The temperature sensor 170 may include a thermistor or other temperature sensing component. The blood pressure sensor 180 may be implanted into the vascular system such as the blood pressure sensors in the leads used by the Medtronic Chronicle™ or use electrical signals from an electrical lead to the carotid sinus like the Rheos™ Baroreflex Hypertension Therapy System™ by CVRx. Such leads could be connected to the VSM for monitoring blood pressure. Another technique is to process the PPG signal to provide short term changes in blood pressure. Finally, there are numerous automated blood pressure cuffs for legs, arms, wrists or fingers that could be worn by the patients and enhanced to telemeter the patient's blood pressure to the VSM 10 or alternately the EXD 50.

The VSM 10 also includes an accelerometer 175 which can be used to monitor patient activity associated with cardiac events and/or other vital signs changes. A clock timing sub-system 130 allows the VSM 10 to record the time and date of data recording and system events detected by the VSM 10 or initiated by the patient through the EXD 50. The VSM 10 can also save various heart signal parameters and/or other vital signs in histogram format and can process these histograms to extract processed histogram data which can be retained to show longer term changes in patient condition. U.S. patent application Ser. No. 10/950,401 by Fischell et al which is included herein by reference, describes the use of histograms and extracted histogram data.

The VSM 10 may also include an internal alarm sub-system designed to alert the patient to the detection of specific events. Similarly, the EXD 50 may also include patient alerting capabilities. The use of such internal and external patient alerting is well described by Fischell et al in U.S. Pat. Nos. 6,609,203, 6,272,379 and 6,468,263 which are incorporated herein by reference.

FIG. 7 is a block diagram of a vital signs monitoring system 100 including an implanted vital signs monitor (VSM) 10, an external device (EXD) 50, a vital signs monitor display 70 and remote diagnostic equipment 80. FIG. 7 shows the components of the external device (EXD) 50 of FIG. 6. The EXD 50 is controlled by the EXD processor 53 with memory 54. The EXD processor 53 is designed to communicate with the implanted VSM 10 through the telemetry sub-system 51 with antenna 52.

An alerting sub-system 56 with acoustic transducer 57 allows the EXD 50 to communicate via audio signals to the patient. Examples of these audio signals include beeps, buzzes and spoken announcements. Examples of the acoustic transducer 57 include small loudspeakers and piezoelectric transducers. The visual display 58 allows the EXD 50 to communicate with the patient using visible information. The visual display 50 may be one or more LEDs that indicate a specific message or in a more sophisticated embodiment, the visual display 50 may have the capability to display text or pictures to the patient. Patient alerts can be triggered by detection of physiological events by the VSM 10 and/or EXD 50 as well as status and confirmation messages associated with the function of the vital signs monitor system.

Control button(s) 55 provide patient control of the EXD 50 and through wireless communication, the VSM 10 as well. Examples of uses of the control button(s) 55 include turning off a patient alert or alarm, initiating data (ECG, PPG etc) capture and storage by the VSM 10, and initiation of a telecom session to offload data from the VSM 10 and/or EXD 50 to the remote diagnostic equipment 80.

The EXD 50 also includes the flash memory interface 59 into which an external flash memory device 65 can be inserted or attached. Examples of such external flash memory devices 65 include Compact Flash cards, memory sticks, Secure Digital (SDIO) cards, Multimedia cards (MMC) and USB thumb drives. The advantage of a removable flash memory device 65 is that they are inexpensive, can store huge amounts of data and can be removed from the EXD and inserted into a personal computer or PDA having the appropriate standardized interface. As the physician's programmer 90 may be a modified laptop computer, use of a USB thumb drive may be the preferred embodiment as all current personal computers have the capability of reading such a storage device.

It is also envisioned that the EXD 50 could include built in non-removable flash memory. In either case, the EXD 50 is designed to allow transfer of stored data through the charging cradle 60 having a telecom interface 62. The telecom interface 62 may be a simple telephone line modem with an RJ-11 jack connected to a phone line, or it may be a wired or wireless Ethernet (TCPIP) interface. A wired Ethernet interface would typically include a RJ-45 jack for CAT5 cable connection while a wireless Ethernet connection could use any current or future standard wireless Ethernet protocol such as 802.11.a, b, g or n. The telecom interface 62 facilitates data transfer to remote diagnostic equipment 80. The remote diagnostic equipment 80 for example, can be part of the infrastructure of a service bureau or a system located in the office of the patient's doctor. In either case, the remote diagnostic equipment would typically include means to allow review of the data collected by the VSM by a medical professional.

The VSM system 100 can perform continuous Holter monitoring by having data stored in the VSM continuously or periodically transferred to the EXD. The resulting data can be offloaded to the remote diagnostic equipment 80 or transferred by removing the flash memory device 65 from the EXD 50 and inserting it into a device designed to process and/or display the data. The VSM display 70 and physician's programmer 90 would include the ability to interface with the flash memory device 65.

The VSM display 70 is a device external to the patient that would typically be used by a medical practitioner to access data stored in the VSM 10 or even the EXD 50. The VSM display 70 can serve as a diagnostic instrument in a doctor's office, emergency room, ambulance, or hospital ward. In some ways the VSM display 50 would function like the medical tricorder envisioned by the creators of the science fiction series Star Trek. It would allow the medical practitioner to quickly see what is going on inside the patient. The VSM display 70 could, for example, be built into a PDA device running Microsoft Pocket PC or Palm OS.

The physician's programmer 90 is designed to program both the VSM 10 and EXD 50 to set up patient vital signs monitoring customized to each patient. It can also offload, process and display data collected by the VSM 10 or transferred from the VSM 10 to the EXD 50. The physician's programmer 90 can also set thresholds for detection of physiological events and specify what data is collected and what patient alerting (if any) is associated with the detection of each event.

It is also envisioned that the EXD 50 could have a replaceable battery instead of the rechargeable battery 65. An alternate embodiment of the EXD 50 would not require a charging cradle but would have the telecom interface 62 built into the EXD 50 itself. In this embodiment the telecom interface could be a modem and RJ-11 phone line jack, a standard Ethernet interface with either wired (RJ-45) or wireless (802.11.a, b, g, or n) connection capability, or the telecom interface could be designed to connect to a cellular data network such as provided in the United States by T-Mobile, Cingular, Verizon or Sprint. The cellular interface would allow wireless transfer of data from the VSM 10 and EXD 50 to the remote diagnostic equipment 80 from any place with access to a cellular data network.

FIG. 8 shows a 20 second trace of the PPG signal 165 which is the output of the PhotoPlesmoGraphy (PPG) sensor 160 of FIG. 6. The VSM 10 of FIG. 6 (as well as the VSM 30 of FIGS. 4A, 4B and 5) is designed to process the PPG signal 165 to track heart and lung function, monitor vascular tone and detect abnormal physiological events such as syncope, edema, and sleep apnea. Although the PPG signal can be collected continually, the power drain makes it more efficient to collect the PPG signal periodically. In each period the PPG sensor 160 of FIG. 6 would be turned on for a preset period of time which corresponds to N heart beats. FIG. 8 shows a 20 second period “P” of PPG signal collection although the period P could be as short as several seconds and as long as several hours.

It is envisioned that the primary information in the PPG signal can be calculated by analysis of the Systolic and Diastolic signal voltages for each peak and valley in the PPG signal. The peak (systolic) voltage for the i^{th} peak in the PPG signal 165 of FIG. 8 is shown as element 166 which is V_{S}(i). The valley (diastolic) voltage for the i^{th} peak in the PPG signal 165 of FIG. 8 is shown as element 167 which is V_{D}(i). The i^{th} peak is separated in time from the (i−1)^{th} peak by the RR interval for the i^{th} peak RR(i) having element number 168 in FIG. 8. It is envisioned that each value of V_{S}(i), V_{D}(i) and RR(i) for all N heart beats during the period P of each PPG signal collection would be retained in memory of the VSM 10 of FIG. 6. If these data becomes too large for the memory 154 of the VSM 10 of FIG. 6 then the data may be transferred to the larger storage available in the EXD 50.

It is envisioned that the PPG signal 165 would be collected and analyzed many times per day. For example, the PPG signal 165 might be collected once per hour for 2 minutes. The resulting data would be time stamped so that the date and time of day would be saved along with the voltages and RR intervals. The PPG signal 165 might also be collected continuously at night to detect sleep apnea. In addition, the PPG signal 165 could be collected following the detection of a cardiac event by the electrical signal processing of the CPU 150 of FIG. 6. For example, the detection of Syncope or Bradycardia could trigger PPG signal collection to correlate the blood oxygen level with the electrical anomalies detected. Changes in vascular tone and heart function will result in changes in the amplitude deviation signal A(i)=V_{S}(i)−V_{D}(i) which can also be calculated and stored by the VSM 10 or EXD 50. The effects of congestive heart failure and other decreases in heart function will result in a decrease of A(i) or the average A_{avg}(N) over all N beats of A(i).

It is also envisioned the amplitude deviation A(i) can be monitored as a function of the RR interval RR(i) where effects of decreased heart function will first be seen at higher heart rates (i.e. shorter RR intervals). For example, 5 histograms corresponding to 5 different ranges of RR interval (or heart rate) could be saved in the memory 154 of the VSM 10 of FIG. 6. These histograms would have perhaps 20 bins corresponding to different values of A(i). Each time A(i) is calculated for the i^{th} beat from the PPG signal 165, the histogram whose range corresponds to the RR interval RR(i) for i^{th} beat will have the bin whose range includes the value of A(i) incremented by 1. Over a histogram data collection period, the mean or median value of amplitude deviation for all beats within a range of RR interval can be calculated. These mean or median values, which are extracted histogram data, can be retained for extended periods of time to identify slow changes in heart and lung function. Fischell et al in U.S. patent application Ser. No. 10/950,401 describes in detail how such histograms and extracted histogram data can be processed by an implantable device.

Another important aspect of the PPG signal 165 is the average signal level V_{A} shown as element 169 in FIG. 8. This signal may be calculated within the VSM 10 or EXD 50 or at a later time by the VSM display 70 or programmer 90 from the peak and valley data collected by the VSM 10. The average signal level V_{A} may also be analyzed to identify maxima V_{IN}(j) and minima VE_{EX}(j) elements 161 and 162 corresponding to inhalation and exhalation respectively of air from the lungs. To save memory space, just the maxima V_{IN}(j) and minima V_{EX}(j) for each breath as well as the breathing cycle time T_{B}(j) for the j^{th} breath during the data collection period P. The CPU 150 of the VSM 10 of FIG. 6 can also calculate the breathing deviation signal B(j)=V_{IN}(j)−V_{EX}(j). Similar to the histograms created for monitoring A(i), one or more histograms of the type envisioned by Fischell et al in U.S. patent application Ser. No. 10/950,401 can be processed and retained by the VSM 10 to track the breathing deviation signal B(j). It is also envisioned that multiple breathing deviation histograms may be processed by the CPU 150 with each of the multiple histograms corresponding to a different range of breathing cycle time T_{B}(j). This technique could compensate for the changes seen as a result of slow or fast breathing thus allowing more effective tracking of lung function over time.

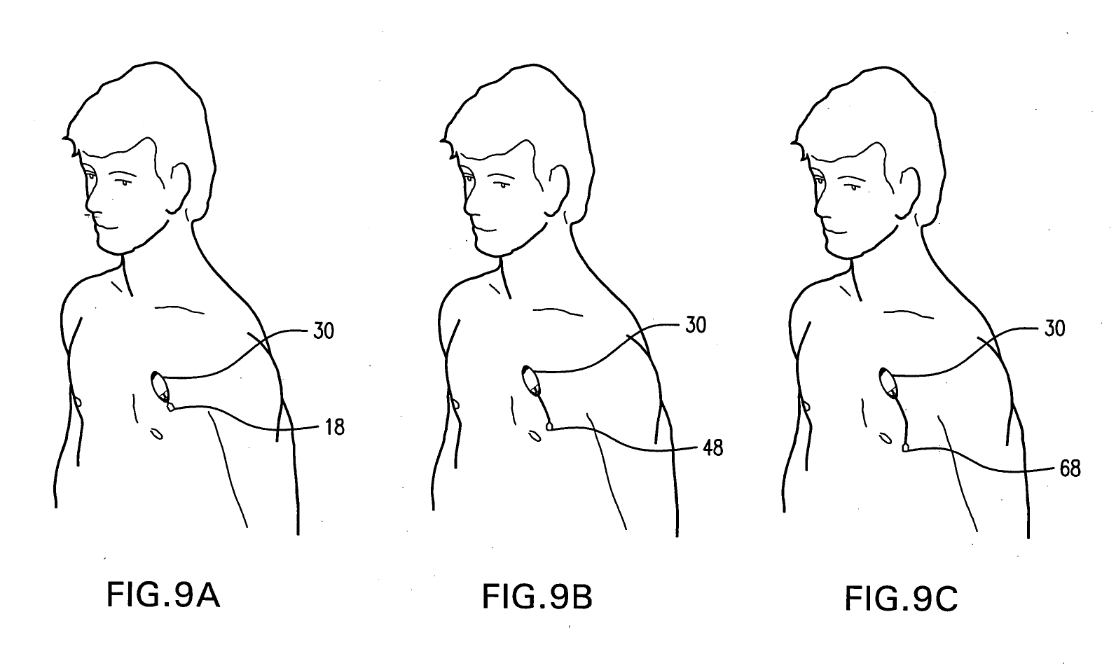

FIGS. 9A, 9B and 9C show three different lead configurations for the present invention VSM 30. In FIG. 9A, the short lead subcutaneous 18 is attached to the VSM 30 and is typically used for applications where only R wave measurements are needed. The short lead 18 is typically less than 1 inch in length. In FIG. 9B, the medium length subcutaneous lead 48 is attached to the VSM 30 creating a longer spacing between the electrodes suitable for measurement of P waves as well as R waves. The medium lead 48 is typically between 1 and 6 inches in length. In FIG. 9C, the long subcutaneous lead is attached to the VSM 30 and is used for applications where electrocardiogram morphology including QRS width, and ST segment levels are to be measured. It is also envisioned that by including a standardized lead connection 34 in the header 33 of the VSM 30, a pacemaker or ICD intracardiac lead or an endocardial lead attached to the outside of the heart can also be used to bring signals into the VSM 30. For example, an intracardiac lead attached to the apex of the right ventricle might provide the best data for ST segment levels associated with ischemia and an endocardial lead attached below the septum between right and left ventricles might provide the best signal for measuring QRS amplitude changes associated with heart transplant rejection.

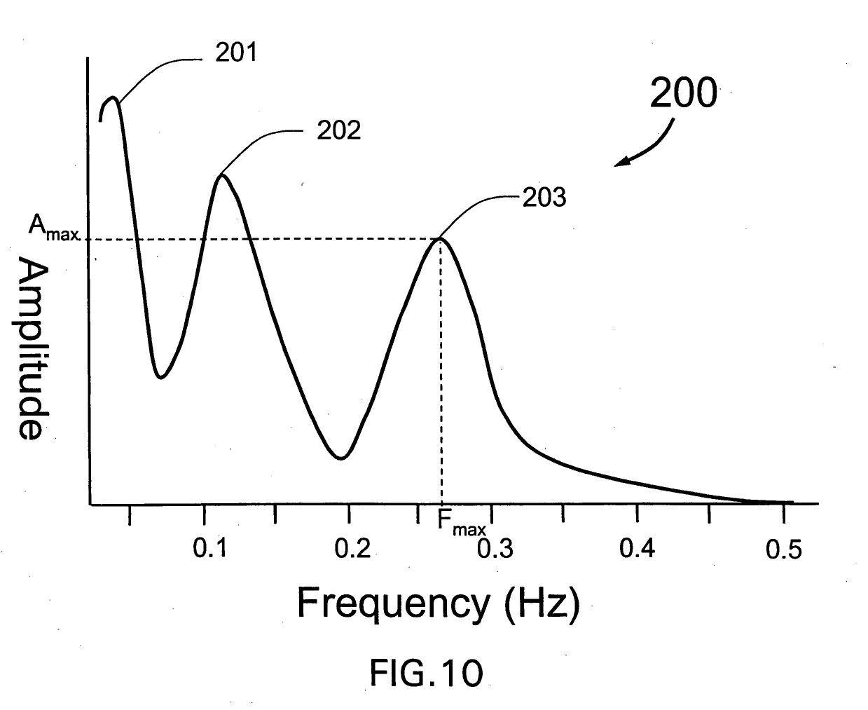

Another important measurement that can be made by the VSM 10 or 30 of FIGS. 1 through 5 is the R-R interval variability. The RR interval variability can be calculated by the VSM 10 or 30 either from the electrical signal sensed by the electrodes 14 and 19 or the PPG signal 165. R-R interval variability measurements can provide information on the autonomic nervous system and the overall health of the heart. Loss of R-R interval variability is often associated with heart failure or a precursor to arrhythmias. Kamath et al in “Power Spectral Analysis of Heart Rate Variability: A Noninvasive Signature of Cardiac Autonomic Function” published in Critical Reviews in Biomedical Engineering, 21(3):245-311 (1993) describe how there are 3 distinct peaks in the power spectrum of R-R interval variability. These peaks shown in the spectrum 200 of FIG. 10 are a low-frequency peak 201 near 0.05 Hz, a mid-frequency peak 202 near 0.1 Hz and a high-frequency peak 203 between 0.3 Hz and 0.5 Hz. The high-frequency peak 203 has an amplitude Aₘₐₓ and a frequency Fₘₐₓ. Kamath further discusses how changes in the levels of these three peaks are correlated with specific bodily functions and disorders. Specifically, the low-frequency peak is linked with vasomotor and/or temperature control, the mid-frequency peak is associated with baroreceptor-mediated blood pressure control and the high-frequency peak is strongly correlated with respiratory sinus arrhythmia.

Measurement of R-R interval variability can be calculated by one or more processors within the VSM 10 or 30 or in the external equipment 60 of FIG. 6. While there are many ways to calculate the R-R interval variability not all are suitable for implementation in an implantable device with limited memory, processing speed and power. For an implantable device, the present invention R-R interval variability measurement can be accomplished as follows. At scheduled times each day, the R-R interval for each beat (RRᵢ) is measured for all but the first of “n” beats occurring during a preset time period “τ”. The R-R interval RRi being the time from the i^{th} R wave to the preceding (i−1)^{th} R wave. The average R-R interval (RR_{avg}) is also calculated for the same preset time period τ. The R-R interval variability ρᵢ for each beat, which occurs at a time tᵢ into the time period τ, is given by the expression
ρᵢ=RRᵢ−RR_{avg}

So for the total time period τ there will be n−1 values of R-R interval variability (you can't calculate R-R interval variability for the 1^{st} beat). From these n−1 values one can calculate the average R-R interval variability ρ_{avg} which provides a gross measurement of the amplitude of R-R interval variability during the time period τ. It is also desirable to measure the power spectrum of the R-R interval variability. To get this, one can collect the raw data of each ρᵢ or RRᵢ and the beat time tᵢ and from this the R-R interval variability power spectrum can be calculated using Fourier analysis methods such as described by Kamath et al. Such methods are easily implemented in the external equipment 60 of FIG. 6 but would be less practical in an implantable device or external device such as the EXD 50 of FIGS. 6 and 7.

Perhaps the most important R-R interval variability analysis can be performed by examining only the high-frequency peak (at approximately 0.3 Hz) strongly correlated with respiratory sinus arrhythmia. To get this information, the R-R interval variability values and times (ρᵢ, tᵢ) and be analyzed using a half-wave analysis technique of the R-R interval variability time history produced by the plot of ρᵢ, vs. time to generate and approximation of the R-R interval variability power spectrum. This plot of ρᵢ, vs. time will have maxima and minima spaced at an interval of between 0.5 and 1.5 seconds. To get an approximate power spectrum using the present invention half wave technique one calculates the amplitude and duration of each half wave where the j^{th} half wave in the plot of ρᵢ, vs. time is the segment of the plot of ρᵢ, vs time between the maximum (maxⱼ) and minimum (minⱼ). For each maximum maxⱼ there are two half waves, a half-wave with amplitude aₖ₋₁ and duration dₖ₋₁ from the preceding minimum minⱼ₋₁ to the maximum maxⱼ and a half-wave with amplitude aₖ and duration dₖ from the maximum maxⱼ to the next minimum minⱼ. The equivalent frequency fₖ of the k^{th} half wave is then given by the expression:
fₖ=¼dₖ
where the frequencies fₖ will be typically between 0.01 Hz and 0.5 Hz.

Using this, the amplitude and R-R interval values become an array of half wave data values (aₖ, fₖ). The values of frequency can then be quantized into a preset number of bins of width δ. For example, to process the high-frequency R-R interval variability peak, the frequencies between 0.2 Hz and 0.5 Hz might be split into 15 bins of width d=0.02 Hz. Thus each of the half wave data values (aₖ, fₖ) are each assigned to a specific frequency bin (those out of range are ignored). Then the average value of amplitude for each frequency bin is calculated. This will produce an amplitude vs. frequency R-R interval variability power spectrum for the high-frequency peak that approximates a true Fourier spectral analysis but does not require the complexity of calculation of a Fourier analysis. This half-wave analysis of the present invention can therefore be implemented within an implantable device such as the VMS 10 or 30 of FIGS. 2 through 7 or in the EXD 50 of FIGS. 6 and 6. The primary information of value from this R-R interval variability power spectrum is the maximum amplitude Aₘₐₓ of the high-frequency peak and the frequency bin Fₘₐₓ at which the high-frequency peak is found. Specifically, these data can be calculated by the VMS 10 or 30 or the EXD 50 at preset times each day. The values can then be used to track the daily cycles and longer term changes in the para-sympathetic autonomic nervous system. It is also envisioned that the system 100 can be programmed to alert the patient if such changes indicate a worsening of the patient's health. Also through the remote diagnostic equipment 80, the system 100 can alert the patient's physician if such changes.

Examples of how changes in the value of Aₘₐₓ can be used include, tracking the daily cycle where Aₘₐₓ increases in the evening and decreases in the morning. A significant change in the difference between the morning and evening values or an overall reduction in time of all the values of Aₘₐₓ can indicate a worsening of the patient's condition.

Various other modifications, adaptations, and alternative designs are of course possible in light of the above teachings. Therefore, it should be understood at this time that, within the scope of the appended claims, the invention can be practiced otherwise than as specifically described herein.

What is claimed is:

1. A implantable medical device for subcutaneous implantation within the body of a human patient the device including:
at least two electrodes for sensing the electrical signals from the patient's heart;
electronic circuitry including digital memory designed to record the electrical signals from the heart; the electronics being housed in a case having a tapered shape to facilitate implantation and removal of the device.

2. The device of claim 1 where the case is tapered in thickness with the ends being thinner than the middle.

3. The device of claim 1 where the case is tapered in width with the ends being thinner than the middle.

4. The device of claim 1 where the case is tapered in both width and thickness.

5. The device of claim 1 further including a detachable subcutaneous lead.

6. The device of claim 5 where the detachable lead is less than 1 inch long.

7. The device of claim 5 where the detachable lead is between 1 and 6 inches long.

8. The device of claim 5 where the detachable lead is greater than 6 inches in length.

9. The device of claim 5 where the detachable lead has exactly one electrode.

10. The device of claim 5 where the detachable lead has two or more electrodes.

11. The device of claim 1 further including a blood oxygen level sensor.

12. The device of claim 11 where the blood oxygen level sensor is a photoplesmography sensor.

13. The device of claim 1 further including a temperature sensor.

14. The device of claim 1 further including means to calculate and store R-R interval variability data.

15. The device of claim 1 further including a blood pressure sensor.

16. The device of claim 1 further including patient alerting means.

17. The device of claim 16 where the patient alerting means is located within the device.

18. The device of claim 16 where the patient alerting means is located external to the device.

19. The device of claim 16 also including patient alerting means located within the device. and a separate patient alerting means external to the device.

20. The device of claim 1 further including telemetry means, the telemetry means being designed to communicate with external equipment at a distance of greater than six inches.

21. A implantable medical device for subcutaneous implantation within the body of a human patient the device including:
a shell housing containing the electronics and battery for the implantable medical device;
at least two electrodes for sensing the electrical signals from the patient's heart where one electrode is located on the shell housing and the other electrode is located on a detachable subcutaneous lead, the detachable subcutaneous lead having one of at least two different lengths.

22. The device of claim 21 where the detachable lead is less than 1 inch long.

23. The device of claim 21 where the detachable lead is between 1 and 6 inches long.

24. The device of claim 21 where the detachable lead is greater than 6 inches in length.

25. The device of claim 21 where the detachable lead has exactly one electrode.

26. The device of claim 21 where the detachable lead has two or more electrodes.

27. The device of claim 21 further including a blood oxygen level sensor.

28. The device of claim 27 where the blood oxygen level sensor is a photoplesmography sensor.

29. The device of claim 21 further including a temperature sensor.

30. The device of claim 21 further including means to calculate and store R-R interval variability data.

31. The device of claim 21 further including patient alerting means.

32. The device of claim 31 where the patient alerting means is located within the device.

33. The device of claim 31 where the patient alerting means is located external to the device.

34. The device of claim 31 also including patient alerting means located within the device and a separate patient alerting means external to the device.

35. The device of claim 21 further including telemetry means, the telemetry means being designed to communicate with external equipment at a distance of greater than six inches.

36. A method of calculating the spectrum of R-R interval variability, the method including the steps of:
a. implanting a medical device within the body of a human patient, the medical device being part of a system including at least one processor designed to measure the timing of beats of a patient's heart from a signal sensed within the patients body;
b. having the at least one processor measure the R-R interval for a multiplicity of beats during the preset time period;
c. having the at least one processor calculate average R-R interval for the multiplicity of beats during the preset time period;
d. having the at least one processor calculate the R-R interval variability as the difference between the R-R interval and average R-R interval for the multiplicity of beats during the preset time period.

37. The method of claim 36 further including the steps:
e. having the at least one processor construct a time history of R-R interval variability;
f. having the at least one processor calculate the amplitude and duration of each half wave formed between maxima and minima of the time history of R-R interval variability.
g. having the at least one processor construct a power spectrum of R-R interval variability from the amplitude and duration data of the half waves.

38. The method of claim 37 further including a step h of having the processor compare the R-R interval variability power spectrum at a given time to the spectrum of R-R interval variability from an earlier time.

39. The method of claim 38 further including a step i of alerting the patient if the comparison indicates a change in the R-R interval variability power spectrum exceeds a preset threshold.

40. The method of claim 39 where the patient is alerted by an alerting signal produced by the implanted medical device.

41. The method of claim 39 where the patient is alerted by an alerting signal produced by the external equipment.

42. The method of claim 25 where the medical device includes electrodes for sensing electrical signals from the human heart, the timing of beats being measured from the electrical signals.

43. The method of claim 36 where the medical device includes a photoplesmography sensor for sensing oxygen levels from the patient's vasculature, the timing of beats of the patient's heart being measured from the signal produced by the photoplesmography sensor.

44. The method of claim 36 where there is at least one processor located in the implanted medical device 45. The method of claim 36 where there is at least one processor located in external equipment 46. The method of claim 45 where there are at least two processors with at least one processor being located in the implanted medical device and at least one processor being located in the external equipment.
